(19) United States
(12) Patent Application Publication
(10) Pub. No.: US 2023/0271109 A1
(43) Pub. Date: Aug. 31, 2023

(54) Method for Accurately Separating and Identifying Oxidized Triglyceride in Frying Oil

(71) Applicant: Jiangnan University, Wuxi (CN)

(72) Inventors: Yuanfa Liu, Wuxi (CN); Xinyu Cao, Wuxi (CN); Yongjiang Xu, Wuxi (CN); Liyang Yuan, Wuxi (CN); Yanpeng Du, Wuxi (CN); Zhaojun Zheng, Wuxi (CN)

(21) Appl. No.: 18/301,388

(22) Filed: Apr. 17, 2023

Related U.S. Application Data
(63) Continuation of application No. PCT/CN2021/098379, filed on Jun. 4, 2021.

(30) Foreign Application Priority Data
Apr. 13, 2021 (CN) 202110395197.3

(51) Int. Cl. C11B 7/00; B01D 15/30

(52) U.S. Cl. CPC C11B 7/0083; B01D 15/305

(57) ABSTRACT

The present disclosure discloses a method for accurately separating and identifying an oxidized triglyceride in frying oil, and belongs to the technical field of detection. In the present disclosure, the structure of the oxidized triglyceride (ox-TG) in the frying oil is identified by mass spectrometry first. It is found that after frying is conducted for 24 h, the ox-TG mainly includes epoxy ox-TG, hydroxyl ox-TG, and aldehyde ox-TG. Thus, the three types of ox-TG are selected as a template molecule to synthesize a surface molecularly imprinted polymer (SMIPs). Then, a polymer completely matched with the ox-TG template molecule in action site and spatial configuration is synthesized, and the specific ox-TG can be separated by using the SMIPs. According to the present disclosure, OXTG-SMIPs prepared by a molecular imprinting technology has good specificity, stability, and affinity, and accurate separation of the ox-TG in the frying oil can be achieved.

### TECHNICAL FIELD

The present disclosure relates to a method for accurately separating and identifying an oxidized triglyceride in frying oil, and belongs to the technical field of detection.

### BACKGROUND

Due to special flavor and taste, fried food is very popular among people. However, oil will constantly undergo reactions in a frying process, and a large number of polar substances that are harmful to the human body are produced. The polar substances refer to a type of substances such as an oxidized triglyceride (ox-TG), free fatty acids, and triglyceride polymers that have higher polarity than normal triglycerides and are generated by a series of chemical reactions such as oxidation, hydrolysis, and polymerization under the condition of heating or frying of frying oil. Because of the polar substances, the function, sensory evaluation, nutritional value, and flavor of the oil are changed. Studies have shown that the most toxic component of the polar substances is the ox-TG. The ox-TG refers to a triglyceride having an oxidized fatty acid chain and a complicated structure and being difficult to analyze and identify.

At present, the polar substances in the frying oil are mainly separated by a silica gel column chromatography method. According to the method, the purpose of separation is achieved based on the polarity difference between polar components and non-polar components. However, only mixed polar components can be obtained by the method, and ox-TG compounds with highest toxicity cannot be individually separated.

### SUMMARY

In the present disclosure, based on a molecular imprinting technology, the structure of ox-TG in frying oil is identified by mass spectrometry first. It is found that after frying is conducted for 24 h, the ox-TG mainly includes epoxy ox-TG, hydroxyl ox-TG, and aldehyde ox-TG. Thus, the three types of ox-TG are selected as a template molecule to synthesize a surface molecularly imprinted polymer (SMIPs). Then, a polymer completely matched with the ox-TG template molecule in action site and spatial configuration is synthesized, and the specific ox-TG can be separated by using the SMIPs. According to the present disclosure, OXTG-SMIPs prepared by the molecular imprinting technology has good specificity, stability, and affinity, and accurate separation of the ox-TG in the frying oil can be achieved.

Specifically, the present disclosure has the following technical solution: a method for accurately separating and identifying an oxidized triglyceride in frying oil. The method includes the following steps:
(a) preparation of a surface molecularly imprinted polymer: first, grafting an outer layer of a substrate material with a functional monomer; adding a template molecule, namely epoxy ox-TG, hydroxyl ox-TG and/or aldehyde ox-TG; then, after the functional monomer is prepolymerized with the template molecule for 1-2.5 h, adding an initiator (azodiisobutyronitrile) and a crosslinking agent (ethylene glycol dimethacrylate) to fix a complex into a polymer network so as to obtain a crosslinked polymer; and finally, subjecting the template molecule in the crosslinked polymer to elution to obtain an oxidized triglyceride-molecularly imprinted polymer (OXTG-SMIPs), where the functional monomer includes any one of methacrylic acid, acrylamide, and 4-vinylpyridine;
(b) preparation of a chromatographic column: loading the OXTG-SMIPs obtained in step (1) into a chromatographic column;
(c) separation with the chromatographic column: adding the frying oil to the chromatographic column, and adding an eluting agent for elution so as to separate the oxidized triglyceride from the frying oil; and
(d) elution of an eluent: sequentially adding eluting agents to the chromatographic column for eluting the oxidized triglyceride, and that is to say, conducting separation in sequence to obtain epoxy ox-TG, hydroxyl ox-TG and/or aldehyde ox-TG.

In an embodiment of the present disclosure, in step (a), the substrate material includes any one or more of silica gel, glucan, and titanium dioxide, and is preferably the silica gel.

In an embodiment of the present disclosure, in step (a), the functional monomer is preferably the methacrylic acid or the acrylamide.

In an embodiment of the present disclosure, when the functional monomer is the acrylamide or the 4-vinylpyridine, specific operation of grafting the outer layer of the substrate material with the functional monomer in step (a) includes:
(1) adding anhydrous toluene to the substrate material, and adding aminopropyltriethoxysilane and pyridine dropwise under stirring, where a molar ratio of the substrate material to the aminopropyltriethoxysilane is 1:8 to 1:5, the mass of the anhydrous toluene is 20-30 times that of the substrate material, and the mass of the pyridine is 2-3 times that of the substrate material; and conducting stirring under the protection of nitrogen at 85-95 ° Cfor 20-24 h, and then conducting drying to obtain an aminoatedsubstrate material; and
(2) adding anhydrous toluene to the product, namely the aminoatedsubstrate material, prepared in step (1), conducting stirring, and adding triethylamine and a functional monomer precursor dropwise (when the functional monomer is the acrylamide, the functional monomer precursor is acryloyl chloride; and when the functional monomer is the 4-vinylpyridine, the functional monomer precursor is dihydroxy-vinylpyridine, and the triethylamine is used as a catalyst), where a mass ratio of the product prepared in step (1) to the functional monomer precursor is 1:1.5 to 1:1.8, the mass of the anhydrous toluene is 20-30 times that of the substrate material, and the mass of the triethylamine is 0.5-2 times that of the substrate material; and after the dropping is completed, carrying out a reaction under the protection of nitrogen for 20-24 h, and then conducting drying to obtain a substrate material grated with the functional monomer.

In an embodiment of the present disclosure, when the functional monomer is the methacrylic acid, specific operation of grafting the outer layer of the substrate material with the functional monomer in step (a) includes: adding anhydrous toluene to the substrate material, conducting stirring, and adding triethylamine and 3-(triethoxysilyl)propyl methacrylate dropwise, where a mass ratio of the substrate material to the 3-(triethoxysilyl)propyl methacrylate is 1:1.5 to 1:1.8, the mass of the anhydrous toluene is 20-30 times that of the substrate material, and the mass of the triethylamine is 0.5-2 times that of the substrate material; and after the dropping is completed, carrying out a reaction under the protection of nitrogen for 20-24 h, and then conducting drying to obtain a substrate material grated with the functional monomer.

In an embodiment of the present disclosure, in step (a), structural formulas of the hydroxyl ox-TG, the aldehyde ox-TG, and the epoxy ox-TG are as shown in Formula I to Formula III, respectively:

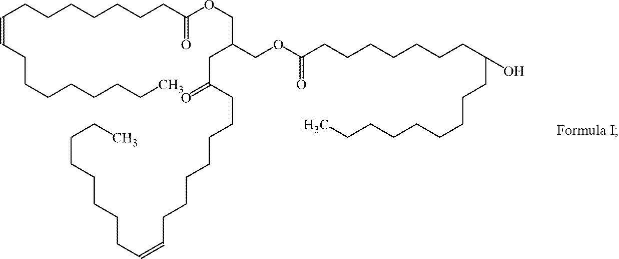 [SMILES: CCCCCCCC/C=C\CCCCCCCC(=O)CC(COC(=O)CCCCCCC/C=C\CCCCCCCC)COC(=O)CCCCCCCC(O)CCCCCCCCC]

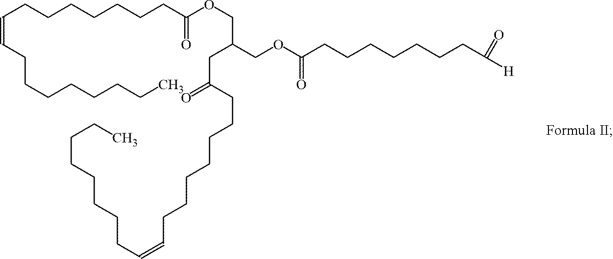 [SMILES: CCCCCCCC/C=C\CCCCCCCC(=O)CC(COC(=O)CCCCCCCC=O)COC(=O)CCCCCCC/C=C\CCCCCCCC]

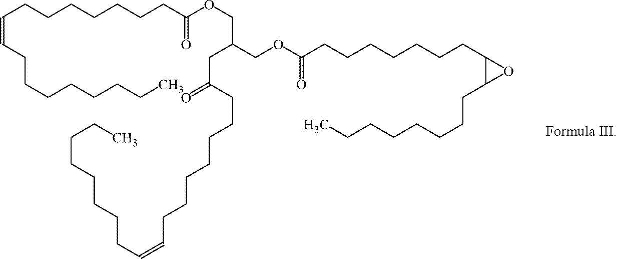 [SMILES: CCCCCCCC/C=C\CCCCCCCC(=O)CC(COC(=O)CCCCCCC/C=C\CCCCCCCC)COC(=O)CCCCCCCC1OC1CCCCCCCC]

In an embodiment of the present disclosure, in step (a), a mass ratio of the substrate material grafted with the functional monomer to the template molecule is 5:1 to 10:1, the use amount of the initiator is 2%-4% (m/m) of that of the substrate material, and the use amount of the crosslinking agent is 1.5-2 times (m/m) that of the substrate material.

In an embodiment of the present disclosure, in step (a), different molecularly imprinted polymers (SMIPs) can be prepared by adding different template molecules as required.

In an embodiment of the present disclosure, when any one of the epoxy ox-TG, the hydroxyl ox-TG, and the aldehyde ox-TG is added, a single-template molecularly imprinted polymer (single-template SMIPs), namely hydroxyl OX-TGMIPs (SMIPs1), aldehyde OX-TGMIPs (SMIPs2), or epoxy OX-TGMIPs (SMIPs3), is prepared.

In an embodiment of the present disclosure, when any two of the epoxy ox-TG, the hydroxyl ox-TG, and the aldehyde ox-TG are added, a double-template molecularly imprinted polymer (double-template SMIPs), namely hydroxyl-aldehyde OX-TGMIPs (SMIPs4), hydroxyl-epoxy OX-TGMIPs (SMIPs5), or aldehyde-epoxy OX-TGMIPs (SMIPs6), is prepared.

In an embodiment of the present disclosure, when the epoxy ox-TG, the hydroxyl ox-TG, and the aldehyde ox-TG are added at the same time, a three-template molecularly imprinted polymer (three-template SMIPs), namely hydroxyl-aldehyde-epoxy OX-TGMIPs (SMIPs7), is prepared.

In an embodiment of the present disclosure, the chromatographic column is preferably a glass sand core chromatographic column with a size ϕ of 45 mm*40 cm.

In an embodiment of the present disclosure, when a single-template molecularly imprinted polymer is prepared, the three types of OXTG-SMIPs, namely the SMIPs1, the SMIPs2, and the SMIPs3, are loaded into the chromatographic column at a mass ratio of 1:1:1 to 1:1:2 in step (b).

In an embodiment of the present disclosure, in step (c), the eluting agent is any one of dimethyl sulfoxide and tetrahydrofuran.

In an embodiment of the present disclosure, in step (c), when a mass ratio of the frying oil to the OXTG-SMIPs is in the range of 1:20 to 1:25, a good separation effect is achieved.

In an embodiment of the present disclosure, in step (c), the frying oil is edible oil used for frying and cooking food in families, restaurants, and industrial occasions.

In an embodiment of the present disclosure, the edible oil refers to animal or vegetable oil used in a preparation process of food.

In an embodiment of the present disclosure, the edible oil includes rapeseed oil, peanut oil, hemp oil, corn oil, olive oil, camellia oil, palm oil, sunflower seed oil, soybean oil, sesame oil, flaxseed oil (linseed oil), flower seed oil, fish oil, algae oil, cottonseed oil, rice oil, grape seed oil, walnut oil, peony seed oil, pig fat, cattle fat, goat fat, and combinations thereof. In an embodiment of the present disclosure, in step (d), the eluting agents sequentially include a mixture of acetic acid and methanol at a ratio of 1:8, a mixture of acetic acid and methanol at a ratio of 1:6, and a mixture of acetic acid and methanol at a ratio of 1:4.

In an embodiment of the present disclosure, a method for accurately separating and identifying an oxidized triglyceride in frying oil specifically includes the following steps:
(a) preparation of a surface molecularly imprinted polymer: weighing 5 g of silica gel as a substrate material, placing the substrate material in a 500 mL round-bottomed three-mouth flask, adding 150 mL of anhydrous toluene, slowly adding 30 ml of aminopropyltriethoxysilane and 10 ml of pyridine dropwise under magnetic stirring at room temperature, and conducting magnetic stirring in a water bath under the protection of nitrogen at 95° C.for 24 h to obtain amino modified SiO₂; after drying is completed, weighing 5 g of the amino modified SiO₂, adding the amino modified SiO₂ to a 250 mL round-bottomed three-mouth flask, adding 100 mL of anhydrous toluene, conducting magnetic stirring for 15 min, slowly adding 8 mL of acryloyl chloride and 5 mL of triethylamine dropwise, and after the dropping is completed, conducting magnetic stirring under the protection of nitrogen for 24 h to obtain SiO₂@acrylamide;
   weighing 5 g of the SiO₂@acrylamide, adding 50 mL of DMSO as a solvent, 0.10 g of azodiisobutyronitrile as an initiator, 8.0 g of ethylene glycol dimethacrylate as a crosslinking agent, and 0.80 g of hydroxyl ox-TG as a template molecule for a reaction for 24 h, and then adding an eluting agent (a mixture of acetic acid and methanol at a ratio of 1:4) for eluting the template molecule so as to obtain SMIPs1;
   and changing the template molecule into 0.80 g of aldehyde ox-TG and 0.80 g of epoxy ox-TG separately to obtain SMIPs2 and SMIPs3 respectively with other operations same as above;
(b) preparation of a chromatographic column: loading the SMIPs1, the SMIPs2, and the SMIPs3 separately obtained in step (1) into a chromatographic column at a mass ratio of 1:1:2;
(c) separation with the chromatographic column: adding the frying oil to the chromatographic column, and adding an eluting agent for elution so as to separate the oxidized triglyceride from the frying oil, where the eluting agent is dimethyl sulfoxide; and
(d) elution of an eluent: sequentially adding eluting agents for eluting the oxidized triglyceride, and that is to say, conducting separation in sequence to obtain epoxy ox-TG, hydroxyl ox-TG and aldehyde ox-TG.

In step (d), the eluting agents sequentially include a mixture of acetic acid and methanol at a ratio of 1:8, a mixture of acetic acid and methanol at a ratio of 1:6, and a mixture of acetic acid and methanol at a ratio of 1:4; and in step (a), structural formulas of the epoxy ox-TG, the hydroxyl ox-TG, and the aldehyde ox-TG are as shown in Formula I to Formula III, respectively:

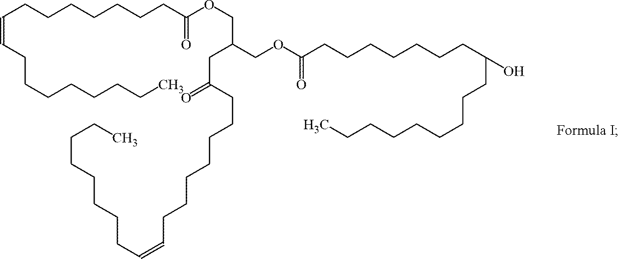 [SMILES: CCCCCCCC/C=C\CCCCCCCC(=O)CC(COC(=O)CCCCCCC/C=C\CCCCCCCC)COC(=O)CCCCCCCC(O)CCCCCCCCC]

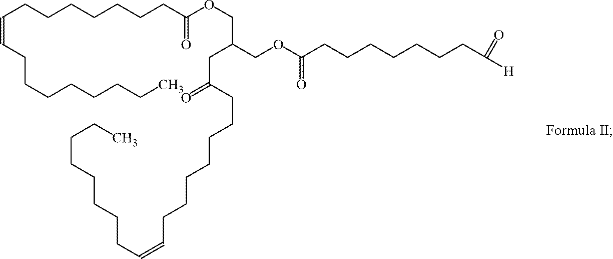 [SMILES: CCCCCCCC/C=C\CCCCCCCC(=O)CC(COC(=O)CCCCCCCC=O)COC(=O)CCCCCCC/C=C\CCCCCCCC]

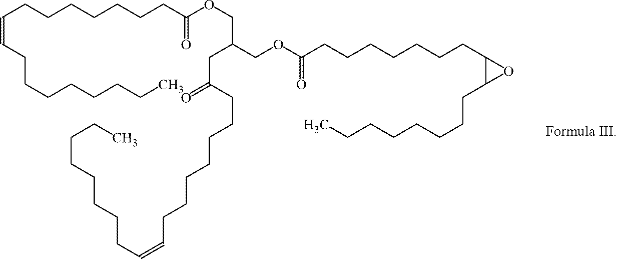 [SMILES: CCCCCCCC/C=C\CCCCCCCC(=O)CC(COC(=O)CCCCCCC/C=C\CCCCCCCC)COC(=O)CCCCCCCC1OC1CCCCCCCC]

In an embodiment of the present disclosure, in step (a), the substrate material is titanium dioxide.

In an embodiment of the present disclosure, in step (a), the acrylamide is changed into methacrylic acid. When the methacrylic acid is grafted, only one step is required for completion, and 3-(triethoxysilyl)propyl methacrylate is used as a raw material.

In an embodiment of the present disclosure, in step (c), the eluting agent is tetrahydrofuran.

The present disclosure further provides application of the method in the field of food.

### The Present Disclosure Has the Following Beneficial Effects

According to the present disclosure, specific OXTG-SMIPs is prepared by a molecular imprinting technology and a Raman spectroscopy technology, the ox-TG in the frying oil can be accurately separated and identified, and the method has the advantages of high affinity, low detection limit and simple operation.

### BRIEF DESCRIPTION OF FIGURES

FIG. 1A is a mass spectrometry EIC images of main ox-TG when frying oil is separately fried for 0 h.

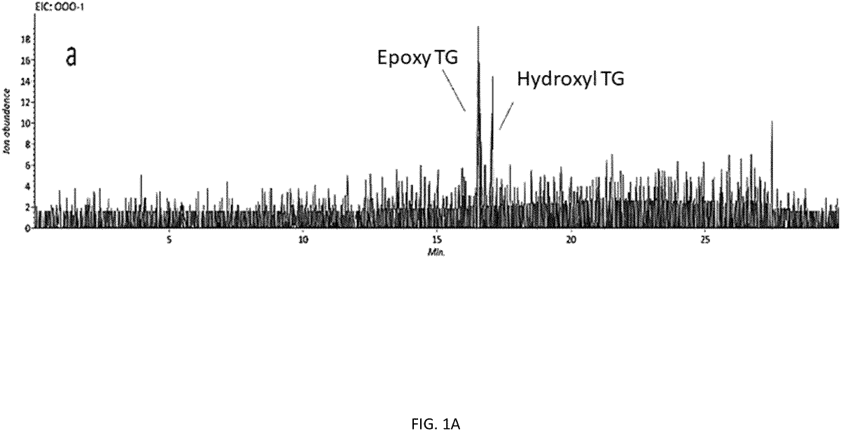

FIG. 1B is a mass spectrometry EIC images of main ox-TG when frying oil is separately fried for 12 h.

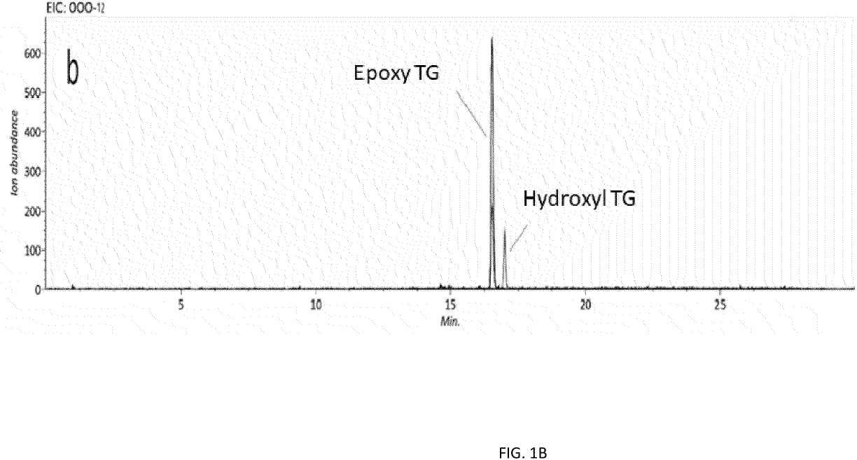

FIG. 1C is a mass spectrometry EIC images of main ox-TG when frying oil is separately fried for 24 h.

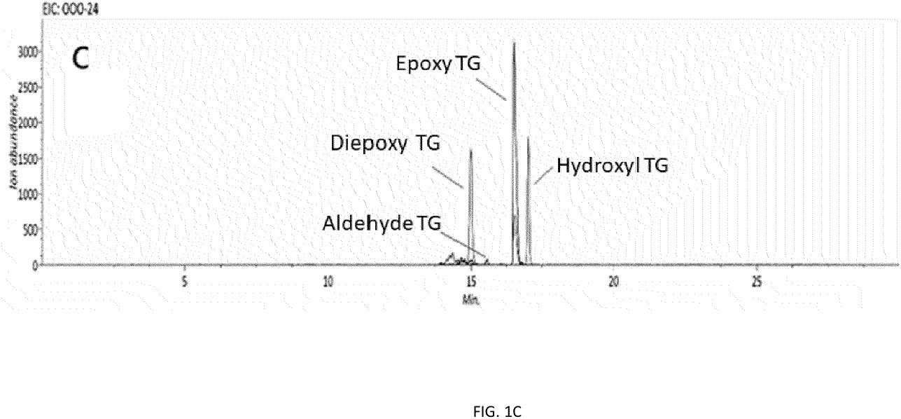

FIG. 2 is a structural formulas of hydroxyl ox-TG, aldehyde ox-TG, and epoxy ox-TG.

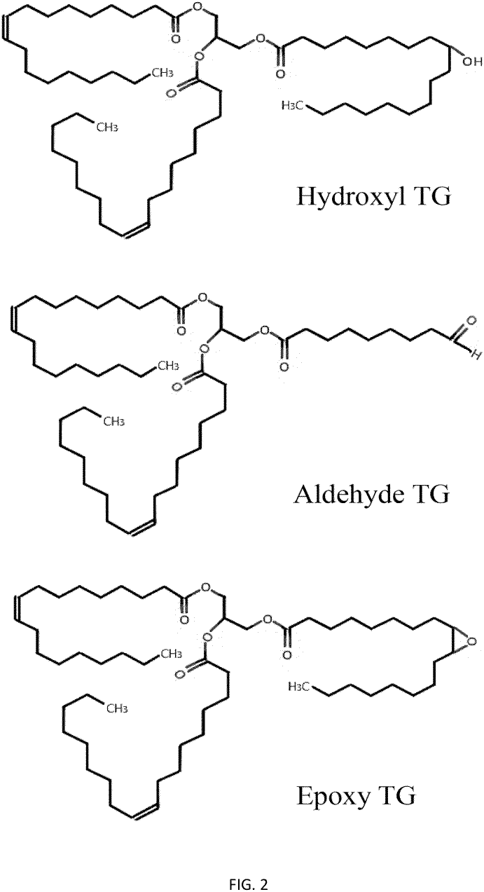

FIG. 3 is a schematic diagram showing the synthesis of OXTG-SMIPs.

FIG. 4 is a diagram showing Fourier infrared spectrum characterization of SiO₂ (a), amino modified SiO₂ (b), SiO₂@acrylamide (c), SMIPs2 (d), and SNIPs (e).

FIG. 5 is a schematic diagram showing the operation of separating ox-TG from frying oil by means of SMIPs.

FIG. 6 is a TLC image showing an ox-TG separation effect of a molecular imprinting method in comparison with a conventional silica gel column chromatography method.

FIG. 7 isa hydrogen nuclear magnetic resonance spectrum image showing an ox-TG separation effect of a molecular imprinting method in comparison with a conventional silica gel column chromatography method.

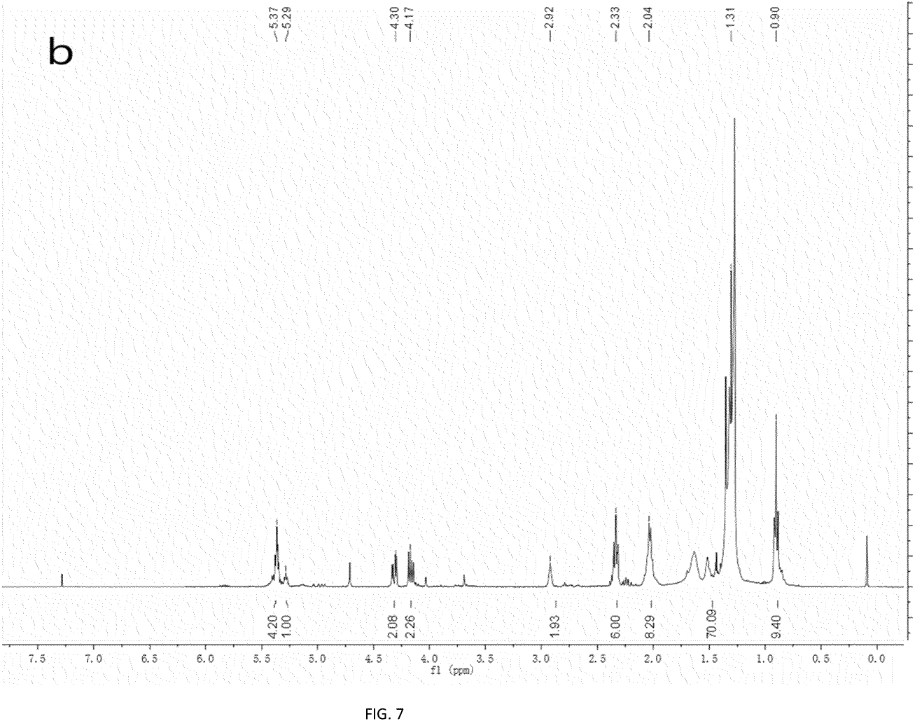

### DETAILED DESCRIPTION

The present disclosure is further described below in conjunction with examples, but the embodiments of the present disclosure are not limited herein.

A determination method and calculation formulas of the RSD, detection limit and recovery rate are as follows: the content of ox-TG obtained after elution is determined by Raman spectroscopy, and the RSD (RSD=standard deviation SD/arithmetic mean X), detection limit (S/N=3) and recovery rate (Recovery=m_{added}/m_{recovered}) are calculated based on the method.

The substrate materials including silica gel, glucan and titanium dioxide, methacrylic acid, acrylamide, and 4-vinylpyridine mentioned in the following examples and comparative examples were purchased from Bailingwei Chemical Reagent Co., Ltd..

### Example 1: Method for Accurately Separating and Identifying ox-TG in Frying Oil

(a) First, 5 g of silica gel as a substrate material was weighed and placed in a 500 mL round-bottomed three-mouth flask, 150 mL of anhydrous toluene was added, 30 ml of aminopropyltriethoxysilane and 10 ml of pyridine were slowly added dropwise under magnetic stirring at room temperature, and magnetic stirring was conducted in a water bath under the protection of nitrogen at 95° C. for 24 h to obtain amino modified SiO₂. After drying was completed, 5 g of the amino modified SiO₂ was weighed and added to a 250 mL round-bottomed three-mouth flask, 100 mL of anhydrous toluene was added, magnetic stirring was conducted for 15 min, 8 mL of acryloyl chloride and 5 mL of triethylamine were slowly added dropwise, and after the dropping was completed, magnetic stirring was conducted under the protection of nitrogen for 24 h to obtain SiO₂@acrylamide.

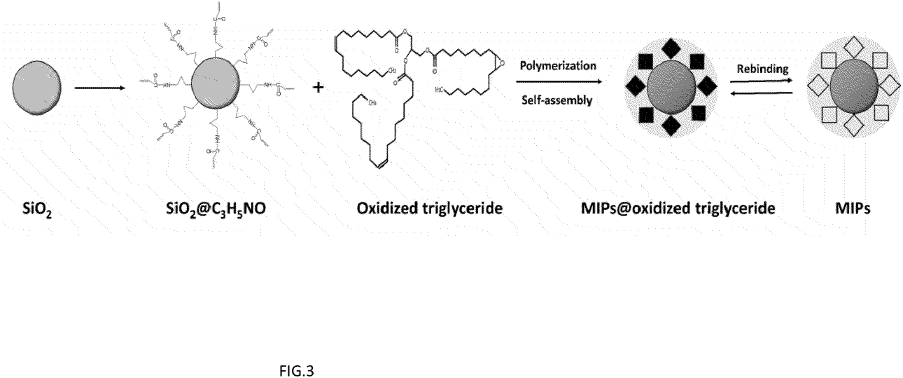

After drying was completed, 5 g of the SiO₂@acrylamide was weighed, 50 mL of DMSO as a solvent, 0.10 g of azodiisobutyronitrile as an initiator, 8.0 g of ethylene glycol dimethacrylate as a crosslinking agent, and 0.80 g of hydroxyl ox-TG as a template molecule were added for a reaction for 24 h, and then an eluting agent (a mixture of acetic acid and methanol at a ratio of 1:4) was added for eluting the template molecule so as to obtain SMIPs1. Another 5 g of the SiO₂@acrylamide was taken, and the template molecule was changed into 0.80 g of aldehyde ox-TG and 0.80 g of epoxy ox-TG separately to obtain SMIPs2 and SMIPs3 respectively with other operations same as above. FIG. 3 is a schematic diagram showing the synthesis of SMIPs. A non-imprinted polymer (SNIPs) was prepared without adding a template molecule, and other preparation steps were the same as above.

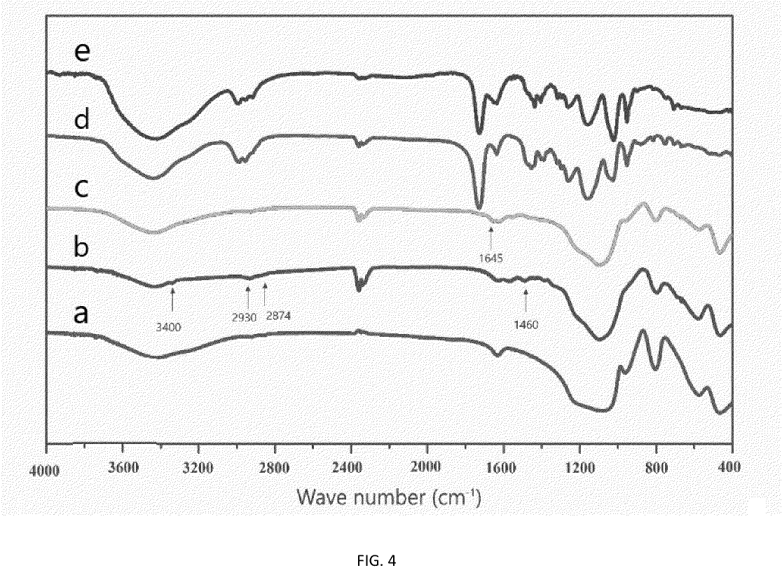

(b) Characterization of the SMIPs was conducted by Fourier infrared spectroscopy. FIG. 4 shows the infrared spectrum of SiO₂ (a), amino modified SiO₂ (b), SiO₂@acrylamide (c), SMIPs2 (d), and SNIPs (e). According to a curve a in FIG. 4, a characteristic absorption peak of Si-O-Si appears at 1,090 cm⁻¹, and it can be observed that a hydroxyl on the surface of an activated silica gel particle appears at 3,500 cm⁻¹, which is conducive to the grafting of a functional monomer on the surface of the silica gel particle. According to a curve b compared with the curve a, a vibration stretching peak of —CH₂— appears at 1,460 cm⁻¹, characteristic absorption peaks of —C—H— appear at 2,930 cm⁻¹ and 2,874 cm⁻¹, and a characteristic absorption peak of the —NH₂ group appears at 3,400 cm⁻¹, indicating that a surface shell of SiO₂ is modified by APTS. According to a curve c, a characteristic absorption peak of the —NH₂ group disappears at 3,400 cm⁻¹, and a characteristic peak of amide appears at 1,645 cm⁻¹, indicating that the amino group is acylated successfully. From curves e and f, it can be clearly seen that the SMIPs and the SNIPs have no significant differences in the position and morphology of absorption peaks and the intensity of characteristic peaks in the spectrum diagrams, and it is proven that a template molecule on MIPs is eluted completely.

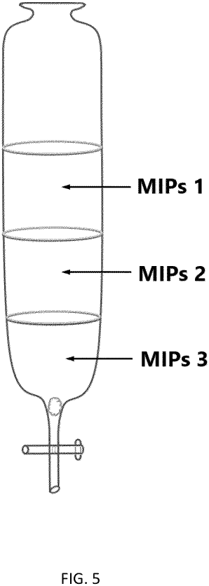

(c) The SMIPs1, the SMIPs2, and the SMIPs3 were sequentially loaded into a chromatographic column (as shown in FIG. 5) at a mass ratio of 1:1:2 (10 g: 10 g: 20 g), 2.00 g of a frying oil sample was added, and then an eluting agent (DMSO) was added for eluting all other components except for ox-TG, where the ox-TG was adsorbed onto the SMIPs.

(d) Sequential elution was conducted: eluting agents including a mixture of acetic acid and methanol at a ratio of 1:8, a mixture of acetic acid and methanol at a ratio of 1:6, and a mixture of acetic acid and methanol at a ratio of 1:4 were added in sequence for elution to obtain hydroxyl triglyceride, aldehyde triglyceride, and epoxy triglyceride.

(e) The content of the ox-TG obtained after elution was determined by a portable Raman spectrometer with potassium thiocyanate (with a characteristic peak at 2,120 cm⁻¹) as an internal standard substance at an excitation light wavelength of 785 nm in a scanning range of 200-400 cm⁻¹, and the test was carried out at room temperature. The epoxy ox-TG has significant characteristic peak values at 810-750 cm⁻¹, 950-840 cm⁻¹, and 1,280-1,240 cm⁻¹. The hydroxyl ox-TG has wide and strong characteristic peaks at 3,700-3,200 cm⁻¹. The aldehyde ox-TG has a significant characteristic peak value at 1,680 cm⁻¹. The steps (c-d) were repeated for five times within one day. The ratio of the peak area of each characteristic peak to the area of an internal standard peak was calculated, the three types of ox-TG were subjected to quantitative treatment, and then the intra-day RSD of the present disclosure was obtained (Table 1).

(f) The steps (c-d) were repeatedly detected at the same time point for five consecutive days. The content of the ox-TG obtained after elution was determined by Raman spectroscopy, and the inter-day RSD based on the method was calculated (Table 1).

(g) The frying oil sample was gradually diluted to obtain various concentrations and then loaded. The steps (c-d) were repeated, and the concentration of the ox-TG in an eluent was determined by Raman spectroscopy. The lowest concentration of the ox-TG that can be detected was recorded, and the detection limit of various types of the ox-TG was obtained (with reference to Table 1).

From Table 1, it can be seen that the maximum RSD_{intra-day} of the present disclosure is 0.6673%, the maximum RSD_{inter-day} is 1.0270%, and the lowest detection limit is 2.0*10⁻⁶ g/ ml. The method has the advantages of high accuracy and low detection limit.

TABLE 1

RSD, detection limit and recovery rate of the present disclosure

| | | | Detection limit | Recovery rate |
|---|---|---|---|---|
| | RSD_{intra-day} (%) | RSD_{inter-day} (%) | (g/mL) | |
| Hydroxyl ox-TG | 0.6673 | 0.8027 | 1.5 × 10⁻⁶ | 94.3±6.5% |
| Aldehyde ox-TG | 0.8572 | 1.0270 | 2.0 × 10⁻⁶ | 87.5±7.2% |
| Epoxy ox-TG | 0.4125 | 0.3152 | 1.5 × 10⁻⁷ | 96.5±4.5% |

### Example 2: Selection of a Substrate Material

The silica gel as a substrate material in step (b) in Example 1 was changed into chitosan or titanium dioxide, and other conditions and parameters were consistent with those in Example 1. Results are as shown in Table 2. It can be seen that the ox-TG in the frying oil can be effectively separated when the substrate material is the silica gel, and the effect is slightly worse when other conditions are used.

TABLE 2

Recovery rate of the method when different substrate materials are used

| | Silica gel | chitosan | Titanium dioxide |
|---|---|---|---|
| Hydroxyl ox-TG | 94.3±6.5% | 87.3±8.9% | 92.4±9.3% |
| Aldehyde ox-TG | 87.5±7.2% | 81.5±15.2% | 82.9±15.0% |
| Epoxy ox-TG | 96.5±4.5% | 88.5±10.5% | 91.1±8.7% |

### Example 3: Selection of a Functional Monomer

The acrylamide as a functional monomer in step (a) in Example 1 was changed into 4-vinylpyridine (dihydroxyvinylpyridine was used as a functional monomer precursor) or methacrylic acid (when the functional monomer was methacrylic acid, the grafting of the functional monomer was required to be completed only in one step, and 3-(triethoxysilyl)propyl methacrylate was used as a raw material), and other conditions and parameters were consistent with those in Example 1. Results are as shown in Table 3. It can be seen that when the functional monomer is the methacrylic acid and the acrylamide, the present disclosure has a better effect.

TABLE 3

Recovery rate of the method when different functional monomers are used

| | Methacrylic acid | Acrylamide | 4-vinylpyridine |
|---|---|---|---|
| Hydroxyl ox-TG | 94.3±6.5% | 91.4±6.7% | 89.4±10.3% |
| Aldehyde ox-TG | 87.5±7.2% | 89.8±9.8% | 86.9±9.8% |
| Epoxy ox-TG | 96.5±4.5% | 95.3±6.0% | 91.8±7.1% |

### Example 4: Selection of an Eluting Agent

The eluting agent (dimethyl sulfoxide) in step (c) in Example 1 was changed into tetrahydrofuran or chloroform separately, and other conditions and parameters were consistent with those in Example 2. Results are as shown in Table 4. When the dimethyl sulfoxide and the tetrahydrofuran are used as the eluting agent, polar components in the frying oil can be effectively separated.

TABLE 4

Recovery rate of the present disclosure when different eluting agents are used

| | Dimethyl sulfoxide | Tetrahydrofuran | Chloroform |
|---|---|---|---|
| Hydroxyl ox-TG | 94.3±6.5% | 90.2±11.2% | 49.9±8.3% |
| Aldehyde ox-TG | 87.5±7.2% | 109.9±14.3% | 44.1±9.5% |
| Epoxy ox-TG | 96.5±4.5% | 97.3±7.8% | 65.3±7.8% |

### Example 5: Selection of the Use Amount of Frying Oil

The mass ratio of the sample (frying oil sample) to the SMIPs in step (c) in Example 1 was changed from 1:20 into 1:15, 1:25, and 1:30 separately, and other conditions and parameters were consistent with those in Example 2. Results are as shown in Table 5. When the mass ratio of the frying oil sample to the SMIPs is in the range of 1:20 to 1:25, a good separation effect is achieved.

TABLE 5

Recovery rate of the present disclosure when different ratios of the sample to the SMIPs are used

| | | | | |
|---|---|---|---|---|
| | 1:15 | 1:20 | 1:25 | 1:30 |
| Hydroxyl ox-TG | 83.4±3.7% | 94.3±6.5% | 82.9±5.6% | 79.6±4.5% |
| Aldehyde ox-TG | 83.8±3.8% | 89.5±7.2% | 92.9±6.2% | 62.5±7.1% |
| Epoxy ox-TG | 96.4±6.0% | 96.5±4.5% | 90.5±10.0% | 87.9±6.3% |

### Comparative Example 1: Comparison With a Traditional Method

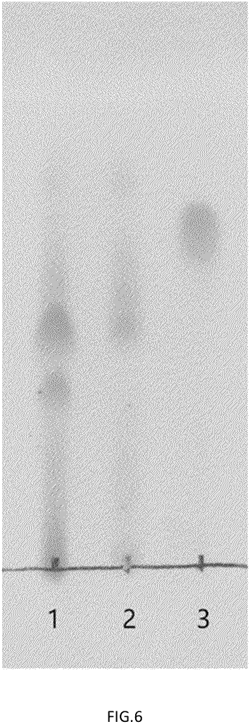

FIGS. 6 (a) is a TLC image of ox-TG separated from frying oil by means of a polar substance (1), column chromatography (2) and SMIPs1 (3). FIG. 6 (b)is a hydrogen nuclear magnetic resonance spectrum image of ox-TG separated from frying oil by means of SMIPs1. According to results, it is shown that three types of ox-TG cannot be accurately separated by means of a traditional chromatographic column, while epoxy ox-TG can be effectively separated from the frying oil by means of the SMIPs1. As described in Example 1, the present disclosure has a recovery rate of 80.3-101.0%, indicating that several types of ox-TG can be accurately separated by using the method.

Although the present disclosure has been disclosed as preferred examples as above, the preferred examples are not intended to limit the present disclosure. Various changes and modifications can be made by any person familiar with the technology without departing from the spirit and scope of the present disclosure. Therefore, the protection scope of the present disclosure shall be as defined by the claims.

What is claimed is:

1. A method for accurately separating and identifying an oxidized triglyceride in frying oil, wherein the method comprises the following steps:
(a) preparation of a surface molecularly imprinted polymer: first, grafting an outer layer of a substrate material with a functional monomer; adding a template molecule, namely epoxy ox-TG, hydroxyl ox-TG and/or aldehyde ox-TG; then, after the functional monomer is prepolymerized with the template molecule for 1-2.5 hours, adding an initiator azodiisobutyronitrile and a crosslinking agent ethylene glycol dimethacrylate to fix a complex into a polymer network so as to obtain a crosslinked polymer; and finally, subjecting the template molecule in the crosslinked polymer to elution to obtain an oxidized triglyceride-molecularly imprinted polymer (OXTG-SMIPs), wherein the functional monomer comprises any one of methacrylic acid, acrylamide, and 4-vinylpyridine;
(b) preparation of a chromatographic column: loading the OXTG-SMIPs obtained in step (1) into a chromatographic column;
(c) separation with the chromatographic column: adding the frying oil to the chromatographic column, and adding an eluting agent for elution so as to separate the oxidized triglyceride from the frying oil; and
(d) elution of an eluent: sequentially adding eluting agents to the chromatographic column for eluting the oxidized triglyceride, and conducting separation to obtain epoxy ox-TG, hydroxyl ox-TG and aldehyde ox-TG.

2. The method according to claim 1, wherein in step (a), the substrate material comprises any one or more of silica gel, glucan, and titanium dioxide.

3. The method according to claim 1, wherein in step (a), the functional monomer is the methacrylic acid or the acrylamide.

4. The method according to claim 1, wherein a mass ratio of the substrate material grafted with the functional monomer to the template molecule is 5:1 to 10:1, the use amount of the initiator is 2%-4% (mass/mass) of that of the substrate material, and the use amount of the crosslinking agent is 1.5-2 times (mass/mass) that of the substrate material.

5. The method according to claim 1, wherein in step (a), structural formulas of the hydroxyl ox-TG, the aldehyde ox-TG, and the epoxy ox-TG are as shown in Formula I to Formula III, respectively:

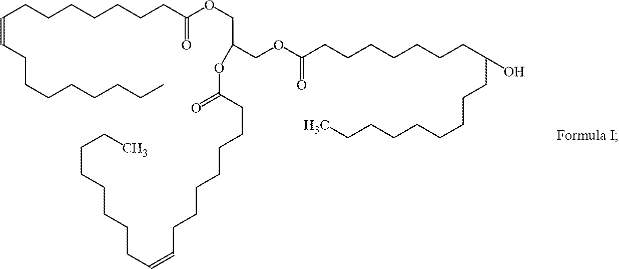 [SMILES: CCCCCCCC/C=C\CCCCCCCC(=O)OCC(COC(=O)CCCCCCCC(O)CCCCCCCCC)OC(=O)CCCCCCC/C=C\CCCCCCCC]

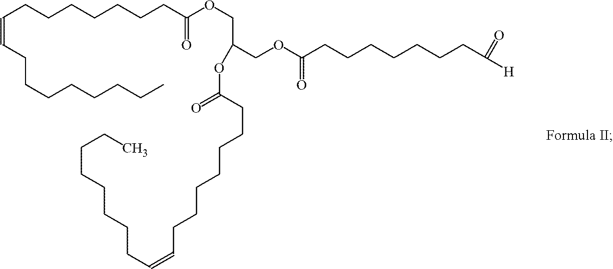 [SMILES: CCCCCCCC/C=C\CCCCCCCC(=O)OCC(COC(=O)CCCCCCCC=O)OC(=O)CCCCCCC/C=C\CCCCCCCC]

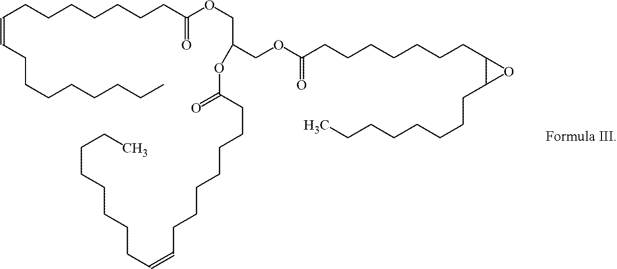 [SMILES: CCCCCCCC/C=C\CCCCCCCC(=O)OCC(COC(=O)CCCCCCCC1OC1CCCCCCCC)OC(=O)CCCCCCC/C=C\CCCCCCCC]

6. The method according to claim 1, wherein when any one of the epoxy ox-TG, the hydroxyl ox-TG, and the aldehyde ox-TG is added, a single-template molecularly imprinted polymer, namely hydroxyl OX-TGMIPs (SMIPs1), aldehyde OX-TGMIPs (SMIPs2), or epoxy OX-TGMIPs (SMIPs3), is prepared.

7. The method according to claim 6, wherein when a single-template molecularly imprinted polymer is prepared, the three types of OXTG-SMIPs, namely the SMIPs1, the SMIPs2, and the SMIPs3, are loaded into the chromatographic column at a mass ratio of 1:1:1 to 1:1:2 in step (b).

8. The method according to claim 1, wherein in step (c), the eluting agent is any one of dimethyl sulfoxide and tetrahydrofuran.

9. The method according to claim 1, wherein in step (c), a mass ratio of the frying oil to the OXTG-SMIPs is 1:20 to 1:25.

10. The method according to claim 1, wherein in step (d), the eluting agents sequentially comprise a mixture of acetic acid and methanol at a ratio of 1:8, a mixture of acetic acid and methanol at a ratio of 1:6, and a mixture of acetic acid and methanol at a ratio of 1:4.

11. The method according to claim 1, wherein the frying oil is edible oil used in families, restaurants, industrial frying, and cooking food.
